(19) United States
(12) Patent Application Publication
(10) Pub. No.: US 2024/0130640 A1
(43) Pub. Date: Apr. 25, 2024

(54) Syringe with Integrated Vein Finder

(71) Applicant: Becton, Dickinson and Company, Franklin Lakes, NJ (US)

(72) Inventors: Praveen Nalawade, Belgaum (IN); Manish Kumar, Bengaluru (IN); Shishir Prasad, Ramsey, NJ (US); Karthik MR, Bangalore (IN)

(73) Assignee: Becton, Dickinson and Company, Franklin Lakes, NJ (US)

(21) Appl. No.: 17/973,183

(22) Filed: Oct. 24, 2022

(51) Int. Cl. A61B 5/15

(52) U.S. Cl. CPC A61B 5/1422

(57) ABSTRACT

A syringe with an integrated vein finder is disclosed, which illuminates a potential venipuncture site of a patient and identifies a vein that is suitable for venipuncture with the syringe, for blood collection or intravenous drug administration. The syringe portion of the system includes a syringe barrel and a pointed-tip needle projecting from the distal end of the barrel for venipuncture. An illumination module is coupled to the syringe barrel; it has an illumination source that projects an electromagnetic beam into an illumination zone distal the syringe's pointed tip, for illuminating and identifying a subcutaneous vein. The integrated vein finder and syringe system is operable by a sole clinician, such as a phlebotomist, who can easily manipulate the syringe and illuminate the venipuncture site with one hand, without assistance of another clinician.

### TECHNICAL FIELD

The present disclosure generally relates to medical syringes with integrated illumination modules, to illuminate and in turn locate a subcutaneous vein, during venipuncture, for blood collection and/or administering drugs to a patient.

### BACKGROUND

Blood-collection venipunctures are frequently performed on patients as a basic medical requirement for most diagnostic tests. While venipunctures are performed frequently by clinicians, such as phlebotomists, some patients have veins that easily collapse or roll, are too thin, or that are difficult to find. These “difficult” veins are most often associated with populations such as children or geriatric patients, but anyone can have them. Patients with difficult veins may require multiple puncture attempts as the phlebotomist attempts to identify alternative puncture sites in the same or in a different vein. Venipunctures are also performed on patients for venous drug administration, with the same challenges to clinicians for identifying veins prior to puncture.

Stationary-mounted and handheld vein finding instruments are marketed for in vitro, external illumination of subcutaneous veins. Some of these devices illuminate potential venipuncture sites of patients with electromagnetic radiation in the infra-red wavelength range. As reported by published researchers, the infra-red beam provides clinicians better visible contrast between veins and surrounding skin tissue than ambient light. The stationary-mounted devices are typically mounted on stands, which require maneuvering the patient relative to a fixed illumination field. Such devices are difficult to utilize for immobile or physically challenged patients in beds or wheelchairs. In large-scale, blood collection sites, phlebotomists test hundreds of patients daily. It would be too time consuming to move and reorient a stationary-mounted vein finding device at a large-scale blood collection site for multiple patients every day. So-called “handheld” vein finding instruments require two clinicians to operate them. A sole clinician must hold and actuate the device with one hand while stabilizing the patient with the other hand. The same clinician cannot simultaneously perform a venipuncture with a syringe because that requires one hand to manipulate the syringe and the other hand to stabilize the patient. Given the need for three hands, two clinicians work together to identify a suitable vein with the handheld vein finding device: one operates the vein finder while the other performs the venipuncture with the syringe. Similarly, during intravenous drug administration by syringe, two clinicians are required to utilize a handheld vein finding device and manipulate the syringe.

### SUMMARY

In a first embodiment, a syringe with an integrated vein finder illuminates a potential venipuncture site of a patient and identifies a vein that is suitable for venipuncture with the syringe, for blood collection or intravenous drug administration. The integrated vein finder and syringe system is operable by a sole clinician, such as a phlebotomist, who can easily manipulate the syringe and illuminate the venipuncture site with one hand, while stabilizing the patient's venipuncture site with the other hand. This integrated system requires little additional clinician training and experience beyond what is needed for routine venipuncture during blood collection or intravenous drug administration. Illuminating the venipuncture site with the integrated system increases likelihood that venipuncture will be completed successfully in a single puncture, without the need to re-attempt puncture at the same or at an alternative site. This integrated system eliminates the need for a second, assisting clinician to find the vein with a separate handheld vein finding device while still reducing the likelihood of performing multiple attempts to puncture a suitable vein without use of the separate handheld device. In some embodiments the system integrates the illumination device within the syringe instrument. In other embodiments, the illumination device is a reusable illumination module that is selectively attached to different syringes as needed. In other embodiments the illumination module is selectively coupled to common, disposable, single-use syringes for blood collection or intravenous drug administration.

One aspect of the present disclosure pertains to an in vivo, subcutaneous vein finding syringe system, comprising a syringe and an illumination module. The syringe portion of the system includes a barrel having a proximal open end, a distal end, a plunger “within the barrel, translatable within the proximal open end of the barrel, and a needle projecting from the distal end of the barrel. The needle has a pointed tip for piercing a subcutaneous vein of a patient (i.e., a venipuncture). The illumination module is coupled to the syringe barrel; it has an illumination source that projects an electromagnetic beam into an illumination zone distal the syringe's pointed tip, for illuminating and identifying a subcutaneous vein of the patient to be pierced by the syringe needle.

In some embodiments, the illumination source projects an electromagnetic beam in the infra-red spectrum with a wavelength in a range from 696 nm to 1000 nm. In other embodiments, the illumination source projects an electromagnetic beam wavelength between 740 nm and 940 nm. In some embodiments the illumination source includes an LED emitter. In some embodiments, the illumination source includes one or more lenses for projecting the electromagnetic beam into the illumination zone. In some embodiments, a lens articulator is coupled to the lens for selectively reorienting the illumination zone relative to the pointed tip.

Another aspect of the disclosure pertains to an in vivo, subcutaneous vein finding syringe system, comprising a syringe and an illumination module. The syringe portion of the system includes a barrel having a proximal open end, a distal end, a plunger “within the barrel, translatable within the proximal open end of the barrel, and a needle projecting from the distal end of the barrel. The needle has a pointed tip for piercing a subcutaneous vein of a patient (i.e., a venipuncture). The illumination module has an enclosure, and an illumination source oriented in the enclosure that projects an electromagnetic beam out of the enclosure into an illumination zone distal the pointed tip, for illuminating and identifying a subcutaneous vein of the patient to be pierced by the syringe needle. The system also includes a selectively detachable mount for coupling the illumination module to the syringe barrel. In some embodiments, the detachable mount further comprises a clamp circumscribing the syringe barrel. In some embodiments, the illumination source includes one or more lenses for projecting the electromagnetic beam into the illumination zone. In some embodiments, a beam articulator is coupled to the enclosure for selectively reorienting the illumination zone relative to the pointed tip. In some embodiments the illumination source includes an LED emitter. In some embodiments, the illumination source projects an electromagnetic beam in the infra-red spectrum with a wavelength in a range from 696 nm to 1000 nm. In other embodiments, the illumination source projects an electromagnetic beam wavelength between 740 nm and 940 nm.

Other aspects of the disclosure pertain to a method for finding an in vivo, subcutaneous vein of a patient and for piercing same with a syringe needle. In practicing this method, a syringe is provided, which includes a barrel having a proximal open end and a distal end, a plunger “within the barrel, translatable within the proximal open end of the barrel, and a needle projecting from the distal end of the barrel. The provided needle has a pointed tip. A provided illumination module is coupled to the syringe barrel. The illumination module has an illumination source that projects an electromagnetic beam into an illumination zone distal the pointed tip. A single clinician selectively orients the illumination zone on a patient's body by moving the syringe; identifies a subcutaneous vein of the patient that is within the illumination zone; and pierces the identified subcutaneous vein with the syringe needle while the punctured vein remains within the illumination zone.

Some embodiments for practicing the method further comprise forming the illumination zone with a lens within in the illumination module, and selectively reorienting the illumination zone relative to the pointed tip by manipulating a lens actuator coupled to the lens, which moves the lens relative to the illumination module. Some embodiments for practicing the method comprise selectively coupling the illumination module to the syringe barrel with a clamping mechanism. Some embodiments for practicing the method further comprise projecting an electromagnetic beam wavelength in a range from 696 nm to 1000 nm into the illumination zone with the illumination source.

### BRIEF DESCRIPTION OF THE DRAWINGS

Exemplary embodiments of the disclosure are further described in the following detailed description in conjunction with the accompanying drawings, in which:

FIG. 1 is a perspective view of an embodiment of a vein finding syringe system, including its syringe and its illumination module;

FIG. 2 is an elevational view of the vein finding syringe system of FIG. 1 illuminating a subcutaneous vein 19V of a patient 15P;

FIG. 3 is a perspective view of the illumination module of FIG. 1;

FIG. 4 is a perspective view of another embodiment of a vein finding syringe system, including its syringe and its illumination module;

FIG. 5 is an exploded view of the yet another embodiment of an illumination module of a vein finding syringe system;

FIG. 6 is a perspective view of a charging station for an illumination module of a vein finding syringe system; and

FIG. 7 is a process flow chart illustrative of a method for finding an in vivo, subcutaneous vein of a patient and for piercing same with a syringe needle.

To facilitate understanding, identical reference numerals have been used, where possible, to designate identical elements that are common to the figures. The figures are not drawn to scale.

### DETAILED DESCRIPTION

Aspects of the vein finding syringe system, including its syringe and its illumination module embodiments disclosed herein facilitate patient venipuncture by a single clinician, such as a phlebotomist, for blood collection. The vein finding syringe system disclosed herein is also facilitates venipuncture and intravenous drug administration by a single clinician. The system disclosed herein facilitates single handed operation of both the syringe and its integrated illumination module without relying on additional clinician assistance that otherwise would be necessary to hold and position an independent, external vein finding device.

The matters exemplified in this description are provided to assist in a comprehensive understanding of exemplary embodiments of the disclosure. Before describing several exemplary embodiments of the disclosure, it is to be understood that the disclosure is not limited to the details of construction or process steps set forth in the following description. The disclosure is capable of other embodiments and of being practiced or being conducted in many ways. Also, descriptions of well-known functions and constructions are omitted for clarity and conciseness.

With respect to terms used in this disclosure, the following definitions are provided. As used herein, the use of “a,” “an,” and “the” includes the singular and plural. In this disclosure, a convention is followed wherein the distal end of the device is the end closest to a patient and the proximal end of the device is the end away from the patient and closest to a clinician.

As used herein, the term “Luer connector” refers to a connection collar that is the standard way of attaching syringes, catheters, hubbed needles, IV tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is generally associated with a syringe and can interlock and connect to the female end located a needle hub.

As used herein, ISO 80369-7:2016 defines a specification for standard Luer connectors including a 6% taper between the distal end and the proximal end. A male standard luer connector increases from the open distal end to the proximal end. A female standard luer connector decreases from the open proximal end to the distal end. According to ISO 80369-7:2016, a male standard luer connector has an outer cross-sectional diameter measured 0.75 mm from the distal end of the tip of between 3.970 mm and 4.072 mm. The length of the male standard luer taper is between 7.500 mm to 10.500 mm. The outer cross-sectional diameter measured 7.500 mm from the distal end of the tip is between 4.376 mm and 4.476 mm. As used herein, the phrases “male standard luer connector” and “female standard luer connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “slope”, “wall”, “top”, “side”, “bottom” and others are used throughout this specification to facilitate understanding, it is not intended to limit any components that can be used in combinations or individually to implement various aspects of the embodiments of the present disclosure.

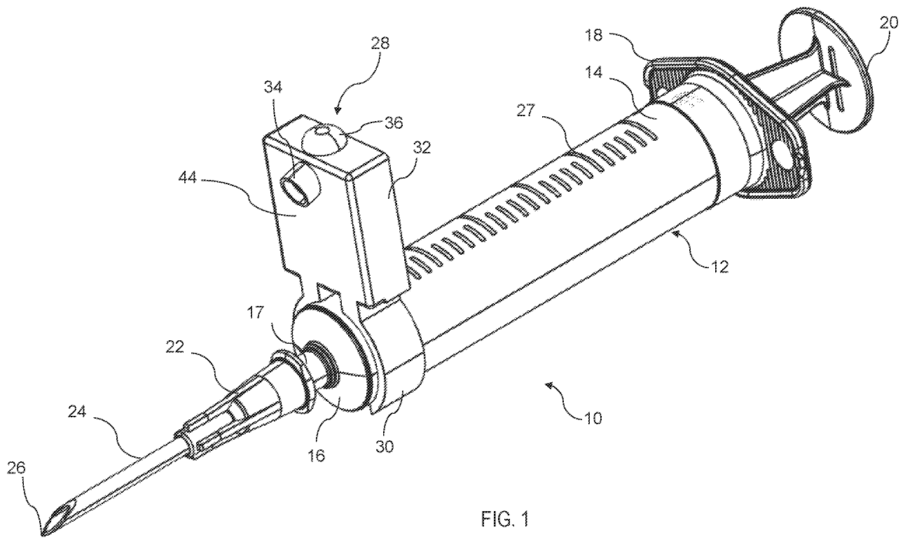

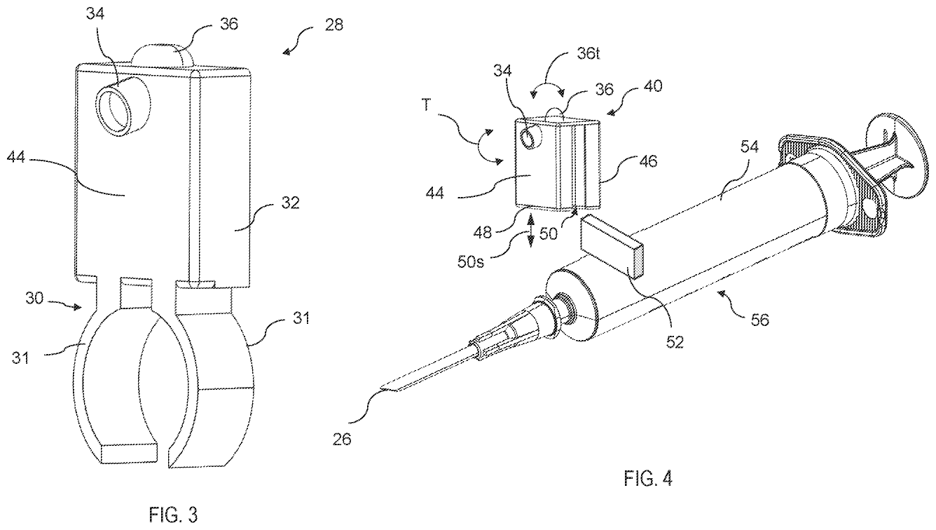

The following non-limiting description demonstrates principles according to one or more embodiments of the disclosure. Referring now to the drawings, a first aspect of the present disclosure is shown in FIGS. 1-3, wherein a vein finding syringe system 10 comprises a syringe 12 and an integrated vein illumination module 28. The syringe 12 is manipulated by a clinician in known fashion to draw blood from or deliver medication intravenously to a subcutaneous vein 19V of a patient 15P (venipuncture).

The syringe 12 includes a barrel 14 having a distal end 16 with a male Luer connector 17, and a proximal end 18, which is open and configured to receive a plunger 20 within the barrel which is translatable within the proximal end of the barrel 14. A needle hub 22, which can be a female Luer connector is coupled to the male Luer connector 17. A needle 24 is coupled to the needle hub 22 and is in fluid communication with a chamber defined within the barrel 14. The needle projects from the distal end of the barrel and has a needle tip 26 for piercing or puncturing the subcutaneous vein 19V of the patient 15P (venipuncture). The syringe 12 includes visual or other indication elements 27 to indicate the position of a stopper (not shown) on a distal end of the plunger 20.

The vein illumination module 28 is selectively coupled to the outer circumference of the syringe barrel 14 by a clamp 30, with a pair of clamp jaws 31 which can be in the form of opposed resilient jaws. The clamp 30 is incorporated within an illumination module housing or module enclosure 32. In other embodiments, the clamp 30 is selectively removeable from the module enclosure 32. In other embodiments, the clamp jaws 31 are selectively biased toward each other by a clamp screw (not shown). A light enclosure 34 is coupled to the module enclosure 32 and transmits an electromagnetic beam into an illumination zone 21Z distal the needle tip 26. The electromagnetic beam is generated by an illumination source within the module enclosure 32. In some embodiments, the light enclosure 34 incorporates the illumination source therein. The illumination source illuminates and identifies a subcutaneous vein 19V of the patient 15P, which is to be pierced by the needle 24. The module enclosure 32 incorporates an articulation mechanism and its articulation knob 36, which can be externally actuated, shown as a track ball, which articulates the illumination zone 21Z relative to the needle tip 26. In some embodiments, the articulation knob is coupled to the light enclosure 34, and the articulation knob can be moved as shown by arrow 36t, which causes the tilt motion T of the light enclosure. The articulation range of motion is shown as a tilt motion T relative to the tip 26, but in other embodiments the articulation range of motion T is a lateral panning motion relative to the tip 26. In some embodiments, the articulation range of motion includes both tilt and panning of the illumination zone 21Z.

Other aspects of the disclosure are shown in FIG. 4, where illumination module 40 has a module enclosure 42 with a front surface 44, a rear surface 46, and a bottom surface 48. A female snap fitting 50 is incorporated in the bottom surface 48 of the module enclosure 42. The snap fitting 50 selectively engages with a corresponding male snap fitting 52 that is affixed to the barrel 54 of a syringe 56, as shown schematically by the arrow 50S. A light enclosure 34 is coupled to the module enclosure 42 and transmits an electromagnetic beam into an illumination zone 21Z distal the needle tip 26. The electromagnetic beam is generated by an illumination source within the module enclosure 42. In some embodiments, the light enclosure 34 incorporates the illumination source therein. The illumination source illuminates and identifies a subcutaneous vein of the patient, which is to be pierced by the needle 24. An articulation mechanism and its articulation knob 36, shown as a track ball, articulates the illumination zone relative to the needle tip 26, as is done in the vein illumination module 28 of FIGS. 1-3. The articulation range of motion is shown as a tilt motion T relative to the needle tip 26, but in other embodiments the articulation range of motion is a lateral panning motion relative to the pointed tip. In some embodiments, the articulation range of motion includes both tilt and panning of the illumination zone. In some embodiments, the articulation mechanism is coupled to the light enclosure 34.

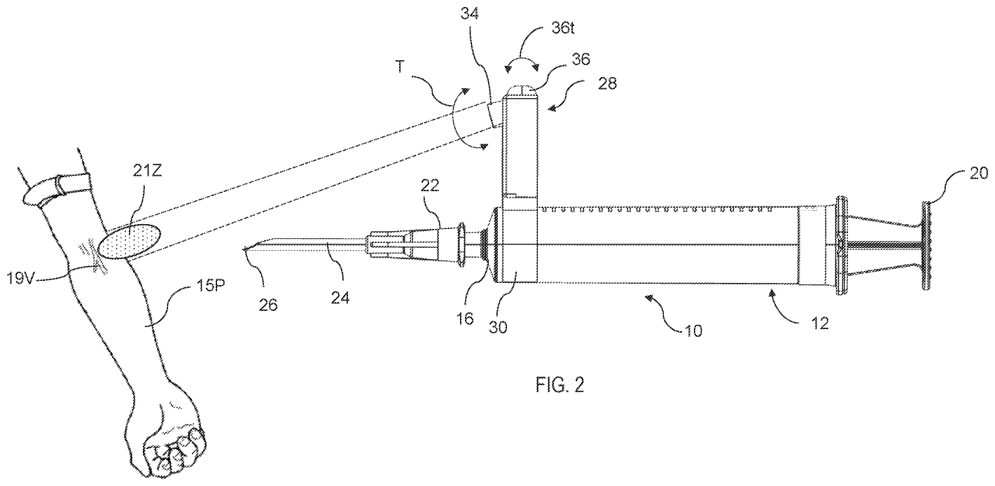

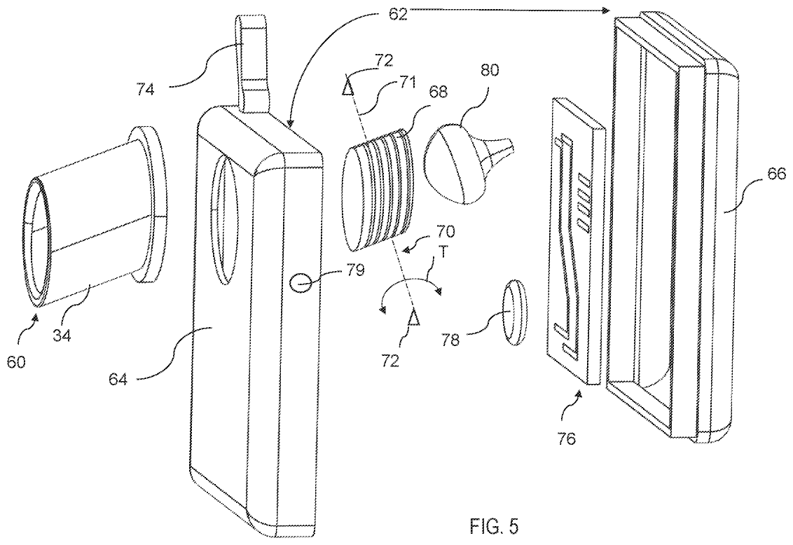

Other aspects of the disclosure are shown in FIG. 5, where illumination module 60 incorporates a split module enclosure 62, with respective front 64 and rear 66 enclosure portions. As shown in the exploded view of FIG. 5, the light enclosure 34 incorporates a single lens or a stack of multiple lenses 68 for focusing the beam of the illumination source in the illumination zone (see, e.g., illumination zone 21Z of FIG. 2). In other embodiments the illumination module has no lens or lens stack for focusing the beam of the illumination source. In other embodiments, the lens or lenses 68 and the illumination source are not retained within the light enclosure 34 and are retained in a fixed position within the module enclosure 62.

A light enclosure tilt mechanism 70, shown schematically, tilts the light enclosure 34 in the range of motion T, about pivot axis 71, and is coupled to pivot mounts 72. The pivot mounts 72 are shown schematically as affixed to the module enclosure 62. The lever knob 74, which can be externally accessible, is manipulated by a clinician to move the illumination zone relative to the needle tip 26. A sole clinician moves the lever knob 74 with the same hand that holds the syringe 12, freeing the other hand to perform other functions, such as stabilize the potential venipuncture site of a patient. The articulation range of motion is shown as a tilt motion T relative to the needle tip 26, but in other embodiments the articulation range of motion is a lateral panning motion relative to the pointed tip. In some embodiments, the articulation range of motion includes both tilt and panning of the illumination zone. In some embodiments, the illumination module has no light enclosure tilt mechanism or external actuation knob.

Referring to FIG. 5, the illumination module 60 retains a printed circuit board 76 within the module enclosure 62 for operable electrical circuit interaction of a battery 78 serving as a power source, and an on/off switch 79 that selectively couples electric power to an illumination source 80. In some embodiments the battery 78 is a disposable battery, such as the button-type battery shown in FIG. 5. In other embodiments, the battery is a rechargeable battery. The illumination source 80 generates the electromagnetic beam that forms the illumination zone. The illumination source 80 comprises a light emitting diode (LED) emitter, which emits electromagnetic radiation, focused by the lens or lenses 68, and projected through the light enclosure 34. More particularly, the illumination source 80 is an LED-IR emitter, which emits electromagnetic radiation in the infra-red wavelength spectrum. In some embodiments, illumination source 80 projects an electromagnetic beam in the infra-red spectrum with a wavelength in a range from 696 nm to 1000 nm. In other embodiments, the illumination source 80 projects an electromagnetic beam wavelength between 740 nm and 940 nm. Various embodiments of the vein illumination modules 28 and 40 employ variations of the components of the illumination module 60.

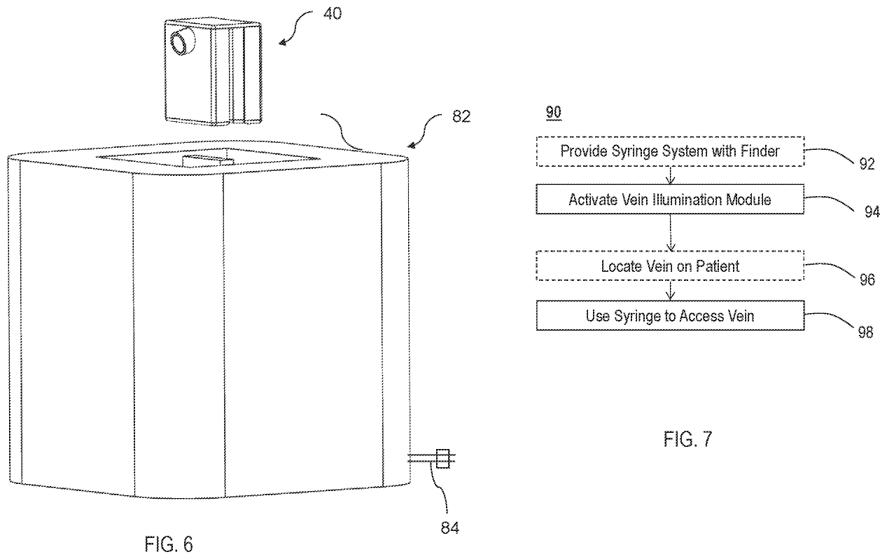

Referring now to FIG. 6, another aspect of the disclosure provides for a rechargeable battery in the vein illumination module 40 for powering the illumination source and a corresponding charging or docking station 82, with the latter powered by a plug-in electrical cord 84. The charging station 82 provides powered to the rechargeable battery of the vein illumination module 40 by direct electrical connection (e.g., USB, mini-USB, micro-USB, connector jack, etc.) or by induction charging transformer. In this manner, the vein illumination module 40 resides in the charging station 82 when not in use. A phlebotomist or other medical clinician retrieves the reusable illumination module and selectively attaches it to a syringe 56 when the need arises to use the vein-finding syringe system for patients with difficult veins. In illumination module embodiments that incorporate a general-use clamp, such as the clamp 30, the phlebotomist can selectively affix the vein illumination module 28 to any type of know syringe by clamping it about the syringe barrel.

An exemplary method of use of the vein finding syringe system 10 and the other embodiments described in this disclosure is shown in the flow chart 90 of FIG. 7. For brevity, the method is described using the vein finding syringe system 10 of FIG. 2, wherein a clinician draws blood from or administer a drug intravenously to a patient. At operation 92 of the flow chart 90, the clinician prepares the syringe 12 by removing it from its packaging. If a vein illumination module 28 is not already affixed to or integrated within the packaged string, it is attached to the syringe barrel 14 by way of the clamp 30 or any other provided syringe/illumination module coupling device. At operation 94 of the flow chart 90, the clinician activates the vein illumination module 28 with an on/off switch, such as the on/off switch 79, thereby activating the illumination source 80, e.g., the LED-IR emitter. When the illumination source is activated, it projects a beam into an illumination zone 21Z distal the needle's needle tip 26. Advantageously, the clinician can reorient location of the illumination zone 21Z relative to the needle tip 26 by manipulating the articulation knob 36. This facilitates use of the vein illumination module 28 as a common module for multiple types of dimensions and geometries of syringes 12. In this manner the clinician has the capability of articulating the illumination zone 21Z in a desired location distal the needle tip 26, no matter what type of syringe is utilized for a specific medical procedure.

At operation 96 of the flow chart 90, the clinician selectively orients the illumination zone 21Z on a patient's body P by moving the syringe and identifies a subcutaneous vein 19V location that is within the illumination zone. The IR wavelength light emitted by the illumination source 80 in the form of an LED-IR emitter on the patient within the illumination zone allows the clinician to view a subcutaneous vein with greater contrast than afforded by ambient light. At any time during the procedure, the clinician can articulate location of the illumination zone 21Z, by manipulating the articulation knob 36. Once a suitable subcutaneous vein 19V is identified with the vein illumination module 28, the clinician punctures the vein with the needle 24 of the attached syringe 12, while the identified vein remains within the illumination zone 21Z, as shown at operation 98 of the flow chart 90.

Upon successful venipuncture, the clinician draws blood from or administers a drug to the patient 15P by manipulating the syringe plunger 20. Clinician manipulation of the vein finding syringe system 10—from initial illumination of the patient with the vein illumination module 28, identification of a suitable vein 19V, orientation of the syringe to puncture the vein, and translation of the syringe plunger 20—are all accomplished with one hand. The clinician's other hand is free to stabilize the patient's venipuncture site or any other task attendant with the medical procedure. A sole clinician can complete successful vein identification and venipuncture of so-called “difficult” veins with the vein finding syringe system 10, without assistance of another clinician. Thus, the vein finding syringe system 10 and the other related embodiments disclosed herein facilitate successful, more comfortable venipuncture for all types of patient veins, by a sole clinician, in less time than required for performing multiple, unsuccessful venipuncture attempts.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. An in vivo, subcutaneous vein finding syringe system, comprising:
a syringe including a barrel having a proximal open end and a distal end, a plunger “within the barrel, translatable within the proximal open end of the barrel, and a needle projecting from the distal end of the barrel, the needle having a pointed tip for piercing a subcutaneous vein of a patient; and
an illumination module coupled to the syringe barrel, having an illumination source that projects an electromagnetic beam into an illumination zone distal the pointed tip, for illuminating and identifying a subcutaneous vein of the patient to be pierced by the needle.

2. The syringe system of claim 1, the illumination module further comprising the illumination source projecting an electromagnetic beam wavelength in a range from 696 nm to 1000 nm.

3. The syringe system of claim 1, the illumination module further comprising the illumination source projecting an electromagnetic beam wavelength between 740 nm and 940 nm.

4. The syringe system of claim 1, wherein the illumination module further comprises:
an enclosure;
the illumination source oriented in the enclosure; and
a lens coupled to the enclosure, for projecting the electromagnetic beam into the illumination zone.

5. The syringe system of claim 4, further comprising a lens articulator coupled to the lens for selectively reorienting the illumination zone relative to the pointed tip.

6. The syringe system of claim 4, the illumination source comprising an LED emitter.

7. The syringe system of claim 6, further comprising the LED emitter projecting an electromagnetic beam wavelength in a range from 696 nm to 1000 nm.

8. The syringe system of claim 6, further comprising a battery power source oriented in the enclosure, for powering the LED emitter.

9. The syringe system of claim 8, the battery power source further comprising a rechargeable battery.

10. An in vivo, subcutaneous vein finding syringe system, comprising:
a syringe including a barrel having a proximal open end and a distal end, a plunger “within the barrel, translatable within the proximal open end of the barrel, and a needle projecting from the distal end of the barrel, the needle having a pointed tip for piercing a subcutaneous vein of a patient;
an illumination module having: an enclosure, an illumination source oriented in the enclosure that projects an electromagnetic beam out of the enclosure into an illumination zone distal the pointed tip, for illuminating and identifying a subcutaneous vein of the patient to be pierced by the needle; and
a selectively detachable mount for coupling the illumination module to the syringe barrel.

11. The in vivo, subcutaneous vein finding syringe system of claim 10, the detachable mount further comprising a clamp circumscribing the syringe barrel.

12. The in vivo, subcutaneous vein finding syringe system of claim 11, further comprising a lens coupled to the enclosure, for projecting the electromagnetic beam into the illumination zone.

13. The in vivo, subcutaneous vein finding syringe system of claim 12, further comprising a beam articulator coupled to the enclosure for selectively reorienting the illumination zone relative to the pointed tip.

14. The in vivo, subcutaneous vein finding syringe system of claim 12, the illumination source comprising an LED emitter.

15. The in vivo, subcutaneous vein finding syringe system of claim 14, further comprising the LED emitter projecting an electromagnetic beam wavelength in a range from 696 nm to 1000 nm.

16. The in vivo, subcutaneous vein finding syringe system of claim 15, further comprising a battery power source oriented in the enclosure, for powering the LED emitter.

17. The in vivo, subcutaneous vein finding syringe system of claim 16, the battery power source further comprising a rechargeable battery.

18. The in vivo, subcutaneous vein finding syringe system of claim 10, further comprising a lens coupled to the enclosure, for projecting the electromagnetic beam into the illumination zone.

19. The in vivo, subcutaneous vein finding syringe system of claim 18, further comprising a beam articulator coupled to the enclosure for selectively reorienting the illumination zone relative to the pointed tip.

20. The in vivo, subcutaneous vein finding syringe system of claim 18, the illumination source comprising an LED emitter.

21. The in vivo, subcutaneous vein finding syringe system of claim 20, further comprising the LED emitter projecting an electromagnetic beam wavelength in a range from 696 nm to 1000 nm.

22. The in vivo, subcutaneous vein finding syringe system of claim 21, further comprising a battery power source oriented in the enclosure, for powering the LED emitter.

23. The in vivo, subcutaneous vein finding syringe system of claim 22, the battery power source further comprising a rechargeable battery.

24. A method for finding an in vivo, subcutaneous vein of a patient and for piercing same with a syringe needle, comprising:
providing a syringe including a barrel having a proximal open end and a distal end, a plunger “within the barrel, translatable within the proximal open end of the barrel, and a needle projecting from the distal end of the barrel, the needle having a pointed tip, and an illumination module coupled to the syringe barrel, having an illumination source that projects an electromagnetic beam into an illumination zone distal the pointed tip;
selectively orienting the illumination zone on a patient's body by moving the syringe;
identifying a subcutaneous vein of the patient that is within the illumination zone; and
puncturing the identified subcutaneous vein with the syringe needle while the subcutaneous vein remains within the illumination zone.

25. The method of claim 24, further comprising: forming the illumination zone with a lens within in the illumination module, and selectively reorienting the illumination zone relative to the pointed tip by manipulating a lens actuator coupled to the lens, which moves the lens relative to the illumination module.

26. The method of claim 25, further comprising the illumination source projecting an electromagnetic beam wavelength in a range from 696 nm to 1000 nm.

27. The method of claim 24, further comprising the illumination source projecting an electromagnetic beam wavelength in a range from 696 nm to 1000 nm.

28. The method of claim 24, further comprising selectively coupling the illumination module to the syringe barrel with a clamping mechanism.
